(19) United States
(12) Patent Application Publication
(10) Pub. No.: US 2013/0288973 A1
(43) Pub. Date: Oct. 31, 2013

(54) Decellularized small particle tissue

(71) Applicant: Edmund Burke, Jersey City, NJ (US)

(72) Inventor: Edmund Burke, Jersey City, NJ (US)

(21) Appl. No.: 13/986,970

(22) Filed: Jun. 20, 2013

Related U.S. Application Data
(63) Continuation-in-part of application No. 13/694,586, filed on Dec. 14, 2012.
(60) Provisional application No. 61/630,561, filed on Dec. 14, 2011.

(51) Int. Cl. A61K 35/12

(52) U.S. Cl. CPC A61K 35/12
USPC 514/17.2

(57) ABSTRACT

A process for producing a decellularized small particle tissue which involves selecting an appropriate tissue starting material from which a decellularized small particle tissue is desired to be prepared, treating the tissue with a decellularizing agent at an acid pH to remove at least a portion of the cellular material therefrom and to yield a product comprising a liquid component and a solid component, subjecting the liquid component and the solid component to a plurality of filters, F₁ through Fₙ, wherein n may be 2, or an integer higher than 2, wherein the pore sizes of the filter F₁ through Fₙ range from 200 microns to 10 Kilo Daltons, yielding filtrates and retentates, recycling either of said filtrates or said retentates or both, either separately or together, to any of steps b) or c) or both, at least one time, and isolating a decellularized small particle tissue from any of steps of the process. Both the product and process are novel.

### CROSS REFERENCE TO RELATED APPLICATIONS

This application is a continuation-in-part of U.S. application Ser. No. 13/694,586 filed Dec. 14, 2012 which claims the benefit of U.S. Provisional Application No. 61/630,561 filed Dec. 14, 2011.

### STATEMENT REGARDING FEDERALLY SPONSORED RESEARCH OR DEVELOPMENT

(Not Applicable)

### REFERENCE TO A SEQUENCE LISTING A TABLE, OR A COMPUTER PROGRAM LISTING APPENDIX SUBMITTED ON A, COMPACT DISC (SEE 37 CFR 1.52(e)(5)

(Not Applicable)

### FIELD OF THE INVENTION

The technical field to which this invention relates is the field of producing and decellularizing tissue. The art abounds with numerous techniques for producing decellularized tissues which are widely useable in such areas as tissue repair, tissue regeneration, wound repair, cell growth media or substrates, filling skin defects and voids, tissue implantation, skin grafting and regrowth, organ repair and organ transplantation and other similar areas.

### BACKGROUND OF INVENTION

The numerous and varied techniques, methods and processes for producing decellularized tissue usually start with tissue from a variety of sources including organs and epithelial tissue and various other sources depending on the needs for the ultimate use of the decellularized tissue. The repair or treatment of various body tissues, such as skin, organs, and the like, has been accomplished using collagen compositions, including tissue membranes comprising collagen, e.g., amniotic membrane, pericardium, dura mater, and the like. A need exists, however, for additional, more versatile compositions that can be used in medical applications in addition, to, or in place of, membranes.

A major difficulty with most of the wide variety of processes and techniques that have been used to decellularize tissue, has been achieving the removal of the cells from the tissue while maintaining in the tissue, beneficial proteins from the starting materials which may be needed in the subsequent use of the decellularized tissue in its medical environment. Similarly, while it is desirable, and in some cases necessary, to retain beneficial proteins, it is also undesirable to retain debris and cellular materials resulting from the decellularizing process since they may cause deleterious effects upon subsequent implantation into the body. Undesirable products which come to mind in this respect are whole cells, nucleic acids, endotoxins and other degradation products.

Decellularized tissues are generally produced in a variety of physical forms, usually as large pieces on which cells may grow. Nevertheless, it has been difficult to produce a small particle tissue while avoiding the difficulties expressed above. Advantages of small particle decellularized tissue include but are not limited to the ability to mold and dry it into a variety of shapes, or to making it injectible, making it filter sterilizable and making it more advantageous for cells to utilize in culture conditions.

The decellularized tissue production techniques normally employed in the art utilize decellularizing agents which are usually oxidizing materials such as hydrogen peroxide, with other ingredients such as a detergent, ethanol, or some other oxidizing material, such as peracetic acid, in the presence of such materials as ferrous salts, copper salts, enzymes and the like. Other methods may be employed using combinations of acids, bases and chelating agents to remove cellular remnants from the starting material. These methods often yield decellularized tissues which are usually unsatisfactory in one way or another, however.

Other techniques involve methods which use harsh materials such as sodium hydroxide or hydrochloric acid under harsh conditions such as extremes in pH, without yielding generally suitable results. Most of these methods are generally unwieldy requiring much manipulation in batch type processes and many of them, especially non-oxidizing acid extractions are not effective decellularizing agents and beneficial proteins remain unextracted to an unsuitable extent. In this regard, the distinction between a mere extraction process and the present inventive decellularizing, particle reduction process should be clear.

In short, it is felt that the present state of the prior art, while sufficient in some cases to remove some cellular materials, leaves much to be desired in terms of the quality of the decellularized tissue, especially in the loss of beneficial proteins remaining after the treatment with the decellularizing agents. The present invention intends to provide a novel process which balances the beneficial proteins remaining in the decellularized, small particle tissue with the removal of materials that are undesirable and yet yield a novel, decellularized, small particle tissue which can be widely used in a variety of indications.

### SUMMARY OF THE INVENTION

The present invention is operable with a variety of starting tissues, such as animal or human placenta, umbilical cord, skin, liver, kidney, spleen and the like, including blood itself. Mainly, we utilize what we term herein as “blood-laden” tissue. By “blood laden” is meant tissue which contains a significant amount of blood after removal of the tissue from the body. A preferred tissue of major use in this invention is the highly vascularized placenta and other highly vascularized organs.

In one aspect of the invention, tissue which is not blood-laden or which has for some reason lost its blood, may be rendered blood-laden by supplying exogenous blood thereto and be useful in the invention. We have found that amounts of blood in the tissue relative to the volume of cells in the tissue can be varied over a wide range to yield suitable results. Indeed, blood itself would be a suitable starting material.

In its general applicability, and according to one broad embodiment, the present invention does not rely specifically on the particular decellularizing agent that is used to treat the tissue. That is, virtually any decellularizing agent or agents that work satisfactorily to produce decellularized tissue convenient to the uses of the practitioner, would be suitable. In this broad embodiment, and in fact, in other embodiments, a critical step lies in a treatment step following the decellularization and size reduction step, which comprises a sequential particle size separation and recycling system. In such decellularization techniques, where blood-laden materials are not usually employed, the invention is effective via the sequential particle size separation and recycling technique described herein. Particle size separation may be accomplished by any means known to those skilled in the art, including e.g., centrifugation and filtration.

Thus, the tissue decellularizing techniques utilized in the broad embodiment of present invention may be any of the techniques that are normally used in the art. Typically, the existing art tissue decellularizing techniques, comprising for example, systems containing hydrogen peroxide and ethanol, or hydrogen peroxide with peracetic acid or a detergent, or enzymes, or deoxycholic acid, or sonication techniques, have been used in the art in some circumstances. In the broad embodiment, following the step in which any decellularizing system may be employed, the present invention utilizes a novel particle size separation and recycling system.

In view of the nature of the reactants of the decellularization and particle size reduction process, and our novel recycling system of the unspent reagents used in the decellularization and particle size reduction step, we are able to use less manipulation of reagents and yet speed up the process of decellularization and particle size reduction. In that regard, it should be noted that the first treatment of tissue in the art whether it includes particle size reduction or not, generally does not consume all of the reagents that are applied to decellularizing the tissue, thus resulting in a very inefficient process. Indeed, when using, for example, a peroxide/peracetic acid mixture as used in the prior art there is sufficient peroxide and acid still remaining in the mixture after treatment to further decellularize tissue remaining in the batch. Yet, in conducting the technique, the art would be content to isolate the treated tissue and expect to have sufficiently purified tissue available to it. The actual result is to the contrary. The resulting decellularized tissue from the art techniques is usually not satisfactory and the process is extremely inefficient and expensive. We have discovered that by using particle size separating methods and parameters that sequentially separate smaller particle sizes, we are able to remove product from the system that is in a desired range of particle size and direct the final effluent back to any upstream stage of particle size separation or the decellularization and particle size reduction step.

### BRIEF DESCRIPTION OF THE SEVERAL VIEWS OF THE DRAWING

The invention will be more completely appreciated and better understood by reference to the accompanying Drawings

FIG. 1 is a schematic diagram depicting various steps involved in the process of the invention

FIG. 2 is a set of 4 schematics depicting variations to the process of the invention that were enacted in an experiment to further clarify the process.

FIG. 3 is a schematic depicting the addition of a third filter into the process of the invention.

FIG. 4 is a schematic depicting the ability to take product from the retentate of the first filter in the process of the invention.

FIG. 5 is a schematic depicting the ability to take product from both the retentate of the first filter in the process of the invention and from the secondfilter in the process of the invention and combining them to make a different product.

FIG. 6 is a schematic flow diagram of various steps involved in the process of the invention. The flow diagram Drawing bears the heading “Injectible Extra Cellular Matrix Process Flow” and is also referred to in paragraph [0023] hereof as “Decellularized Small Particle Tissue Process Flow Using 2 Filters” to emphasize the particular embodiments shown therein.

FIG. 7 is a photograph showing the cross section of a histological section of the product when the first filter retentate and the second filter retentate are mixed in combination.

FIG. 8 is a photograph showing the cross section of a histological section of the product when the first filter retentate is used as a laminate over the second filter retentate.

### DETAILED DESCRIPTION OF THE INVENTION

The invention relates to the preparation of a novel, decellularized small particle tissue. Both the product and the process for making it are novel. The decellularized small particle tissue of the invention can be injectible when the tissue is reduced to a certain particle size and dispersed in a suitable liquid although it may be used in a solid form as well. The term “small particle”, as used herein, is meant to apply to extremely small size particulates of tissue or the tissue components. When present in a liquid vehicle, the novel product is injectible into various body locations. Used as a final small particulate form, it is also useable in many locations on the body and can be used as a void filler, for example. These aspects will be apparent when the description of preparation is considered.

The process of the invention starts with an appropriate blood-laden source of tissue to be decellularized. The term “blood-laden” is meant to be descriptive of any tissue which retains a residual amount of blood as part of the tissue starting material to be decellularized, bearing in mind that in some cases blood itself could be a starting tissue. Additionally, extraneous blood can be allowed to drip off, leaving tissue containing a suitable amount of residual blood. Thus, our decellularizing reaction can be said to be one which is carried out on appropriate tissue in the presence of an amount of blood effective in the decellularizing treatment, without regard to the actual amount of blood associated with the tissue. One skilled in the art will quickly determine an amount which leads to optimum results in carrying out the process on the particular tissue chosen by the user. In some cases, blood can be added to tissue deemed to be insufficient in the amount of blood present.

In the usual decellularizing process, the tissue is treated first with a decellularizing agent, then washed and dried to produce the final product. Many and various decellularizing agents have been used. In one broad aspect of the invention, any decellularizing agent may be employed, since it is believed that the benefits of the invention are more pronounced, at least in this one aspect, when the sequential recycling steps employed following the decellularizing step, as described below, are employed.

In this regard, the first step of the invention, in the broadly described aspect, is submitting in a reaction vessel tissue which has preferably been minced and homogenized, to any decellularizing technique, whether or not there is blood present in the tissue. Thus, we are here speaking of any tissue and any decellularizing technique in the first step of the broad aspect of the invention. Appropriate adjustment in the decellularizing agents will be made when insufficient blood remains in the tissue or additional blood is added if it is desired to employ the blood-laden embodiment. There results a partial removal of cellular components, and other debris in a process which tends to be incomplete and inefficient.

In our inventive process, the entire reaction product mixture obtained as described above, is passed through a particle size separation system comprised of a series of stages, at least more than one stage, designed to capture sequentially smaller particle sizes, the effect of which is to permit unreacted decellularized reagents and tissue to continue their decellularizing and particle size reduction effect while at the same time capturing only the smallest decellularized tissue product, depending on the parameters of the particle size separation system. The effluent (smaller size range of separated particles) from the particle size separation system or any stage thereof, is repeatedly recycled to either or both of the decellularizing and particle size reduction step, to any previous stage of the particle size separation system, to cause repeated decellularization and particle size reduction. The retentate (larger size range of separated particles) of the particle size separation can likewise be recycled upstream to any point in the process. This can be seen more effectively by reference to the flow diagram submitted herewith.

As noted above, in the broad concept of the invention, one may employ any suitable decellularizing agents followed by the repetitive recycling through any suitable size separation system particle size. A preferred embodiment of the invention, however, is one where the starting tissue is blood-laden tissue as aforementioned and the decellularizing agent is hydrogen peroxide (H₂O₂). A preferred size separation system is a plurality of filters, i.e., at least two filters, having sequentially smaller filter sizes. In general, the pore size of the filters of the filtering step range down, for example, from about 200 microns or higher, but preferably below about 100 microns to 0 microns or so, to tenths of microns and most preferably, to 100 Kilo Dalton to 10 Kilo Dalton. At these sizes, the tissue is considered to be of small particle size within the meaning of the present invention. This is preferably accomplished in a plurality of filter stages of at least two filters stages of different pore sizes. The ultimate lowest size is preferably below 50 Kilo Dalton, but a suitable decellularized tissue product may be obtained at a final filter stage of about 100 Kilo Dalton. A variety of filters may be used such as bag filters, cross flow membrane filters, and other typical small pore size filters.

A preferred tissue is blood-laden placenta. We have discovered that the use of the novel, repetitive, recycling system of the invention with the blood-laden tissue enables the use of hydrogen peroxide (H₂O₂) alone, i.e., without the added use of an adjunctive material such as peracetic acid, a detergent, copper salts e.g., copper chloride, or ferrous salts, e.g., ferrous sulfate, or other adjunctive decellularizing agents. One may, if desired, use any of such additional materials, but their presence is not necessary to achieve the results of the invention. The advantage of eliminating the need for additional decellularizing agents is at once apparent. Costs are reduced for one, and the process is much easier to automate if reactants need not have to be used and replenished. There is much less manipulation than in processes using more reactants, in that there are fewer residual materials that need to be washed out.

The temperature at which the decellularizing process of the invention is performed is suitably from about 2° C. to room temperature or slightly above. Temperatures ranging from 5° C. to about 30° C. are suitable, but temperatures high enough to cause gel formation of the collagen structure of the tissue should be avoided. The pH of the process is suitably on the acid side, ranging from 2.5 to 7 and preferably 3 to 5, to produce suitable results.

After the process operations are completed, the product is isolated by any of a variety of techniques, including simple drying of the product at slightly elevated temperatures above room temperature, or lyophilizing of the product, or any such method. Raising the pH before or after lyophilization to from 6 to about 8, facilitates this procedure. The final product may be sterilized in various ways if desired. Preferably, the final product also is dried to a moisture content of less than 80%

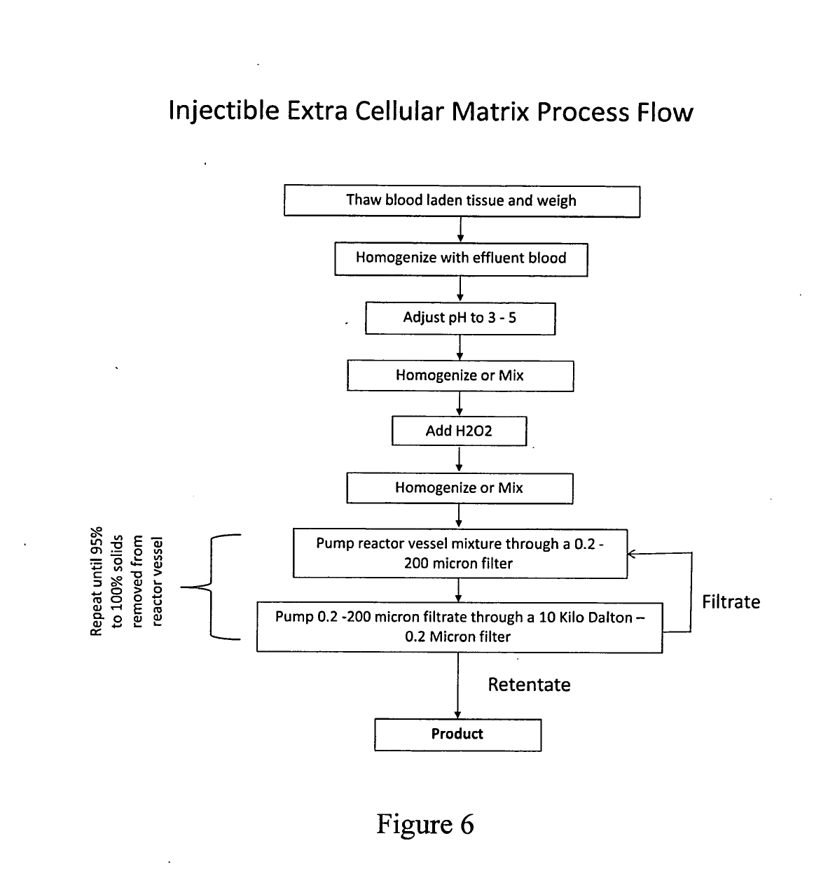

Reference to the flow diagram, FIG. 6, will illustrate general aspects of the invention. The flow diagram headed “Decellularized Small Particle Tissue Process Flow Using 2 Filters,” gives a general schematic flow diagram of the various steps involved in the process of the invention to produce the small particle tissue. An important consideration of the invention is that the reaction scheme employs the decellularization and low pH treatment steps simultaneously or at approximately the same time. In the first step, the blood-laden tissue, which is usually stored frozen, is thawed and then homogenized along with effluent blood to reduce the larger sized tissue into smaller pieces. In this regard, tissue may be cut into pieces of, for example, 50 mm or lower to provide a conveniently handled medium.

Next, the pH is adjusted to, for example, approximately 3 to 6 and then the mixture further mixed, blended, or homogenized in a reaction vessel, and the pH preferably maintained between 4 and 6. The mixture is then treated with hydrogen peroxide and the product mixed for an appropriate time, usually two to twenty-four hours to decellularize and reduce the particle size of the tissue.

Thereafter, the reactor vessel mixture is passed through a first filter of generally 200-0.2 microns and the filtrate then passed through a 0.2 micron to a 10 Kilo Dalton filter to produce more filtrate and retentate.

The filtrates from either or both of the first and second filters are recycled repetitively to the reaction vessel until from about 80% to most, if not all, of the solids are removed from the reaction vessel. In addition, the filtrate from the second filter may be recycled to the first at will and as desired. The ultimate product will be the retentate from the second or last filter.

With regard to the filtering and recycling steps, while the flow diagram shows two filters, the progression through the filter pore size range of 200 microns down to 0.2 microns may be achieved in more than 2 stages. Three or four filters are usually acceptable with the pore sizes decreasing from 200 microns or larger sizes down to 10 KD gradually from the first to the last filter. The product is then isolated using normal techniques, such as by air drying at elevated temperatures above room temperatures or preferably by lyophilizing as indicated below.

While the 200 micron level has been referred above, a larger pore size may be selected for the first filter to provide somewhat more facile processing. The lower end of the range may be any achievable, but for practical reasons, including ease of processing, the lower range pore size is conveniently from about 10 to 50 KD.

From the foregoing, we can describe the process of the invention as comprising the following:
Process 1. A process for producing a decellularized small particle tissue which comprises the following steps:
   (a) Selecting an appropriate tissue starting material from which a decellularized small particle tissue is desired to be prepared,
   (b) treating the tissue with a decellularizing agent at an acid pH to remove at least a portion of the cellular material therefrom and to yield a product comprising a liquid component and a solid component,
   (c) Subjecting the liquid component and the solid component to a plurality of filters, F₁, F₂ and Fₙ, wherein n may be 0, or an integer higher than 0 wherein the pore sizes of the filters F₁, F₂ and Fₙ range from about 10 Kilo Daltons to about 200 microns, yielding filtrates and retentates,
   (d) Recycling either of said filtrates or said retentates or both, either separately or together, to any of steps b) or c) or both, at least one time,
   (e) Isolating a decellularized small particle tissue from any of steps b) or c).

Other particle size separation techniques, such as centrifugation, and other filtering media may be used in place of the filters set forth above. Preferred embodiments are those processes wherein any one, or any combination of the following may be employed in the Process 1 or in processes derived from Process 1.

Process 2. Process 1 wherein a) is a blood-laden placenta or a blood-laden tissue.

Process 3. Any one or more of processes 1 and 2 wherein a) is a blood-laden placenta and b) is hydrogen peroxide.

Process 4. Any one or more of processes 1 through 3 wherein the plurality in step c) is at least 2 filters.

Process 5. Any one or more of processes 1 through 4 wherein the pore size of any filter that is not the sequentially last stage filter is between 0.2-200 microns, wherein at about 0.2 microns and below, the product is considered filter sterilized and does not require additional sterilization or the addition of antibiotics.

Process 6. Any one or more of processes 1 through 5 wherein the pore size of the sequentially last stage (smallest pore size) filter, is between 10 Kilo Dalton-0.2 microns.

Process 7. Any one or more of processes 1 through 6 wherein the pH of step b) is preferably from 3-5.

Process 8. Any one or more of processes 1 through 7 wherein the tissue in step a) is homogenized.

Process 9. Any one or more of processes 1 through 8 wherein the reaction product in step b) is homogenized,

Process 10. Any one or more of processes 1 through 9 wherein filtrate from any filter that is not the sequentially last stage filter (smallest pore size) is passed to the next filter that has the sequentially smaller pore size.

Process 11. Any one or more of processes 1 through 10 wherein retentate from any filter is passed back to the decellularization and size reduction vessel or combined with the filtrate of any upstream filter or both.

Process 12. Any one or more of processes 1 through 11 wherein filtrate from the sequentially last stage (smallest pore size) is recycled to any filter or to the decellularizing and size reduction step a) or both.

Process 13. Any one or more of processes 1 through 12 wherein the isolating step e) is conducted at a pH higher than any of the previous steps b), c) or d).

The novel compounds of the invention may be characterized as the product which is produced from any of processes 1 through 13 or its variations as set forth above and has the characteristics set forth below.

The decellularizing and particle size reduction process of the present invention is effective to produce a product which retains beneficial proteins from the starting tissue to a greater extent than has been achieved in the prior art processes.

The particle size separation system may be any system which effects a particle size reduction such as centrifugation, or filtering systems, for example, provided the size reduction and recycling steps as recited herein are used. The resulting biomaterial composition of the present invention is comprised of tissue that has been treated with a decellularizing agent and then passed through a particle size separation system comprised of a series of stages designed to capture sequentially smaller particle sizes. The effect of this is to permit unreacted decellularized reagents and tissue to continue their decellularizing and particle size reduction effect while at the same time removing as effluent only the smallest decellularized tissue product and soluble reaction media depending on the parameters of the particle size separation system or any stage thereof. The latter stage involves recycling to either or both of the decellularizing and particle size reduction steps to cause repeated decellularization and particle size reduction while retaining the desirable elements of the decellularized tissue in suitable amounts. The retentate (larger size range of separated particles) of any stage of the particle size separation system can likewise be recycled upstream to any point in the process.

As a result, we are able to obtain a novel product capable of being characterized as follows:

A biomaterial comprising tissue that has previously been treated with a decellularizing agent, in a process wherein said tissue has been treated in a particle size separation system capable of separating particle sizes in the range of from about 10 Kilo Daltons to about 200 microns and further comprises the following constituents presented below. The amount of the constituents may be expressed in at least two ways. We prefer to express them here as percent by weight on the basis of the starting biomaterial tissue as one measure and as a percent by weight of the constituent based on the total dry weight of the biomaterial product. Using these parameters, the constituents have the following concentrations:

As a percentage by weight of the dry starting biomaterial tissue concentration:

Collagen, at least 40% and preferably from 60% to 70%;
Elastin, at least 50% and preferably from 70% to 90;
Laminin, at least 10% and preferably from 12% to 21%;
Fibronectin, at least 30% and preferably from 40% to 60%;
ds DNA of less than 10% to 15% and preferably less than 5%; and

As a percentage of the dry weight of the final biomaterial product:

Collagen of at least 30% and preferably from 40% to 50%,
Elastin of at least 2% and preferably from 4% to 5%,
Laminin of at least 0.025% and preferably from 0.045% to 0.01%,
Fibronectin of at least 0.10% and preferably from 0.12% to 0.3%,
Glycosaminoglycan of at least 0.2% and preferably from 0.3% to 0.5%,
ds DNA less than 0.1%, and
Endotoxin less than 0.1% EU/mg.

The utility of the final product may be exemplified by:

a material to heal wounds, to culture cells, as a bone filler, to fill cartilage and tendon,

a material to activate cells prior to application or injection, as a material to activate or evoke a cellular response in vivo,

a material or construction of manmade scaffolding,

further treatment to effect viral inactivation such as pH swings, detergent washing etc.,

filling syringes for use in dermatological procedures,

lyophilizing into or forming as sheets or sponges for wound coverings,

spray drying as particles for cell growth media,

cross-linking to act as filters for plastic surgery, bone void fillers and the like, and

preparing various shapes as above for multiple various uses in the medical arts.

The following Examples will further demonstrate additional aspects and embodiments of the invention.

### Example 1

This Example sets forth the materials and methods used in Examples 2-12.

Hydrogen peroxide used was from Acros Orrganics
2N NaOH and 2N HCL were from Fisher Scientific
Homegenizer used was a OMNI brand model GLH
Endotoxin analysis was done by USP method <85>
Samples for protein analysis were digested in a pepsin—acetic acid solution.
Collagen content was determined by Sirius Red method from Chondrex, Inc.
Fibronectin content was determined by the QuantiMatrix human fibronectin ELISA kit from Milipore.
Laminin content was determined by the QuantiMatrix human fibronectin ELISA kit from Milipore.
Glycosaminoglycan content was determined by the Blyscan Assay from Biocolor inc.
Elastin content was determined by the Fastin Assay from Biocolor, inc.

### Example 2

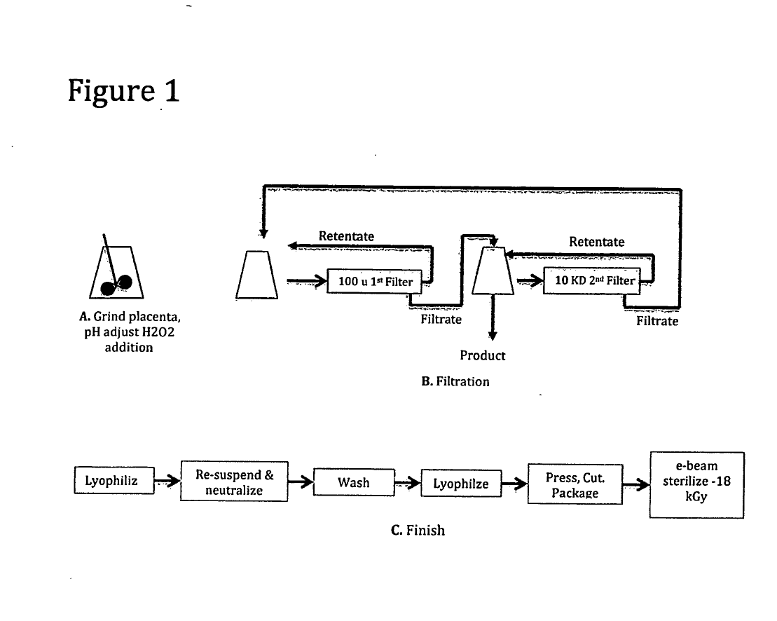

The following Example demonstrates a method of making decellularized small particle tissue from human placenta. A schematic flow of this method can be found in FIG. 1.

Human placenta including the attached umbilical cord and attached amnion and chorionic membrane were obtained from a normal birth. The tissue was obtained after blood was allowed to gravity drain from the cord into a bag for separate purposes. The placenta tissue was stored at −72 degrees C. until all appropriate viral testing and donor history could be reviewed by a qualified medical director in order that it could be released for research and development.

### Grinding, pH Adjust and H2O2 Addition

772 grams of tissue was thawed overnight in a refrigerator and brought to room temperature on the day of processing. It was cut into small pieces (<3 cm in diameter), re-suspended in approximately 1.2 liter sterile water and then homogenized with a hand held homegenizer. A sample of untreated tissue was taken at this time to be used as a control as well as to determine the solids weight of the slurry. A solids measurement was taken via loss on drying method and revealed the solids content to be 7.4%.

While mixing at approximately 500-1000 rpm, the tissue slurry was then adjusted to a pH of about 3.2 with 2N HCL and 2N NaOH if required and mixed for an hour.

Then 3.5 g 30% by weight H2O2 per gram of dry tissue was added slowly over an hour. The slurry was mixed for 10 minutes, and then re-suspended to approximately 6 liters with sterile water.

### Filtration

The slurry was continuously passed through a 1st filter (100 micron in this case) via low pressure mechanical agitation. The permeate from the 100 micron filter was then transferred to a separate vessel where it was continuously passed through passed through a 2^{nd} filter (10 Kilo Dalton (KD) in this case). The permeate from the 2^{nd} filter was then passed back to the 1^{st} filter retentate allowing for further decellularization and particle size reduction. After approximately 30 liters of 2^{nd} filter filtrate was passed back through to the 1^{st} filter retentate, the first filter filtrate became clear indicating that the process could no longer break down the 1^{st} filter retentate. The process was stopped. At this time approximately 90% of the starting material was captured as 2^{nd} filter retentate and approximately 10% of the starting material was captured by as first filter retentate.

### Finish Processing

The 2^{nd} filter retentate material was lyophilized until dry and then re-suspended in 2-3 liters sterile water. The slurry was pH adjusted to between 6.5 and 7 with 2N NaOH and then concentrated by passing through a clean filter (0.5 u in this case) down to 500 mls. This was done two more times to wash the product. The washed slurry was then lyophilized again at approximately 15 mg dry tissue per cm2. The dried sheet was pressed into a pad approximately 500 microns thick. The pad was cut into 3×3 cm squares, packaged and sealed in a foil pouch and sterilized by e-beam irradiation at >18 kGy.

The resulting product had the following characteristics: The collagen content of the product as a percentage of dry material was equal to approximately 45%. The endotoxin content of the product was equal to approximately 0.0185 eu/mg tissue

### Example 3

A human placenta was processed similar to the decellularization and particle size reduction process of Example 2 and FIG. 1 except for the following changes.

The weight of the placenta on removal from the freezer was 465 grams.
The cut up placenta was homogenized with a Waring type blender.
The solids content of the ground unprocessed tissue material was 5.2%.
After the H202 addition, the slurry was mixed for an hour prior to filtration.
After filtration, approximately 85% of the starting material was captured as 2^{nd} filter retentate and approximately 15% of the starting material was captured by as first filter retentate.
The filter used in finish processing step to wash the product had a 10 KD pore size.

The resulting product had the following characteristics:

The collagen content of the product as a percentage of dry material was equal to approximately 53% which is equal to 63% of the collagen content of pre-processed thawed and ground placenta as a percentage of dry material.
The DNA content of the product as a percentage of dry material was approximately 0.25% which is equal to an approximately 71% reduction of the DNA content of pre-processed thawed and ground placenta as a percentage of dry material.
The fibronectin content of the product as a percentage of dry material was equal to approximately 0.14% which is equal to approximately 71% of the fibronectin content of pre-processed thawed and ground placenta as a percentage of dry material.
The laminin content of the product as a percentage of dry material was approximately 0.05% which is equal to approximately 40% of the laminin content of pre-processed thawed and ground placenta as a percentage of dry material.
The elastin content of the product as a percentage of dry material was equal to approximately 4.3% which is equal to approximately 76% of the elastin content of pre-processed thawed and ground placenta as a percentage of dry material.

When the product was used as an additive to standard mesenchymal stem cell culture media as a means to culture rat mesenchymal stem cells in a 3 dimensional collagen gel matrix, the result was an increase in proliferation within 72 hours of between 3 and 5 times that over a control that was cultured in identical conditions except that no placenta extracellular matrix was added.

### Example 4

To further elucidate the beneficial characteristics of the product, samples from Example 3 were tested for their ability to enhance orthopedic soft tissue regeneration.

Achilles tears are devastating injuries, especially to athletes [1, 2]. Unlike ACL injuries and other orthopedic conditions, to date Achilles repair patients have an unpredictable outcome with respect to return to play/function. The surgery itself has minimally evolved over the last 50 years. This is clearly an area which needs more dedicated fundamental investigation and novel therapeutic approaches to improve outcomes.

Treatment protocols for Achilles injuries continue to remain the subject of much debate. There have been several studies which suggest the non-operative treatment may be the clinical equivalent of surgical Achilles repair [3-5]. These studies suggest that early functional rehabilitation may be the key for both surgical and non-surgical treatment options. However, these studies also suggest that the re-rupture rate after non-operative management exceeds that of operative care [3-5]. Furthermore, isolated gastroc-soleus strength testing has also been shown to be improved with surgical management versus conservative treatment [6]. This is balanced by the higher complication rates in those treated surgically [7]. Many studies have shown that at 1 and 2 years post-operatively, the functional outcome of the affected extremity never approaches that of the contralateral, un-injured side [5]. Strength deficits remain as well as calf girth, and persistent discomfort.

In the elite level athlete this discrepancy is further magnified [8]. To date, both management options have not succeeded in predictably returning athletes to a pre-injury level [9]. A significant need exists for improve the surgical repair of these injuries to optimize outcomes.

Regenerative medicine involves the process of replacing or regenerating human cells tissues or organs to restore or establish normal function. The use of natural scaffolds to enhance soft tissue regeneration represents a promising application of regenerative medicine in the field of orthopedics. Cells, scaffolds and the local environment comprise a regenerative triad. The complex interplay between these three key components of tissue regeneration is at the forefront of tissue engineering research and development. Scaffolds play a pivotal role in soft tissue regeneration. Scaffolds provide physical support for cells, thus offering geometric stability, they allow for localization of cells to a specific area of pathology, and they have the ability to incorporate insoluble and soluble signals, such as integrins for cell adhesion and growth hormones for gradual release.

Placental biomaterial is an attractive substrate for use as a scaffold for a number of reasons. It contains numerous natural growth factors, many of which have been shown to enhance tissue repair. It contains a large amount of extracellular matrix, which includes collagen, elastin, laminin, and proteoglycans. It also has natural antibacterial and anti-inflammatory properties.

The purpose of this study was to investigate the properties of decellularized, lyophilized placental biomaterial as both a scaffold and metabolism enhancer for tendon cells. Tenocytes were cultured with placental material, and subsequent tissue specific analyses were performed to evaluate the effects on cell metabolism and tissue specific gene expression.

### Methods:

Experimental Design:

This was an in vitro study of rat tenocytes cultured with lyophilized placental biomaterial in the culture vessel [Exp. group] or culture media alone as a control [Control group] to analyze the effects of placental biomaterial on cell metabolism and gene expression.

Quantitative RT-PCR:

Following harvest at the designated experimental time points cells were harvested and extracted for RNA using the High Pure RNA Isolation Kit (Roche Applied Science) at 24, 48, and 72 hours.

All samples were analyzed in triplicate using quantitative real-time, reverse transcriptase polymerase chain reaction (qRTPCR) using Eurogentec chemistry and the Universal Probe Library (Roche Applied Science). Collagen type I [Col1A1] and scleraxis were primers used to assess tendon gene expression. All RT-PCR runs were normalized with GAPDH, HPRT1, and RPL13A reference genes, designed through Roche RealTime Ready Designs for rattus norvegicus. Calculations were performed using the 2^{-ΔΔCT} method, as described by Arocho A, et al (2006).

Metabolic Studies:

160K rat tenocytes were seeded in each well of six well culture plates with standard growth media for each time point. Cells were allowed 24 hours to attach to the plate. After 24 hours, Placental biomaterial was added to half of the culture plate. On Days 1, 2, 3 and Day 7 cell proliferation and collagen synthesis were determined via uptake of tritiated thymidine (H3-Thy) and tritiated proline (H3-Pro), respectively. To measure cell proliferation, a pulse media composed of 50 μl H3-Thy/100 ml media was added to the culture well 24 hours prior to the experimental time point. Cells were washed with phosphate buffered saline to remove unincorporated isotope followed by cell lysing and measurement of incorporated isotope via scintillation counting. Collagen synthesis measurement by H3-Pro uptake was performed with pulse media of 250 μl H3-Pro/10 ml media added to the culture well 4 hours prior to the experimental time point. All scintillation counts were normalized by DNA content. Data is expressed as cpm/ug DNA. Further preparation and scintillation counting was similar to H3-Thy. Statistical Analysis: Data was analyzed utilizing the unpaired Student's t-test. Statistical significance was defined as p<0.05.

Results:

Tenocytes cultured with placental material demonstrated a progressive, significant increase in collagen type I and scleraxis gene expression at 1, 3 and 6 days This increase in gene expression was 6 and 8.5 fold above control by day 6 for collagen type I and scleraxis respectively. Cell proliferation as measured by incorporation of tritiated thymidine reached significant increase over control by one week in culture. In a similar pattern, collagen synthesis was also significantly enhanced by co culture with placental biomaterial.

Conclusion:

The use of placental biomaterial resulted in a significant improvement in tendon cell metabolism. Both collagen synthesis and cell proliferation were stimulated by the presence of this new biomaterial in their culture conditions. We also observed a significant upregulation of the scleraxis gene, which is important in preserving the tendon phenotype. The significant upregulation of collagen type I correlated well with the observed downstream increase in collagen protein synthesis. This study demonstrates the potential effectiveness of this biomaterial to promote a strong metabolic in tendon tissue which could significantly improve the healing rate when injured. We envision using this novel biomaterial as a circumferential wrap employed during surgical repair of ruptured Achilles tendons. Further studies will focus on in vivo application of this concept in a preclinical model of tendon repair.

### LITERATURE CITED

1. Kujala, U. M., S. Sarna, and J. Kaprio, Cumulative incidence of achilles tendon rupture and tendinopathy in male former elite athletes. Clin J Sport Med, 2005. 15(3): p. 133-5. PMC 15867554
2. Maffulli, N., et al., Achilles tendon ruptures in elite athletes. Foot Ankle Int. 32(1): p. 9-15. PMC 21288429
3. Gwynne-Jones, D. P., M. Sims, and D. Handcock, Epidemiology and outcomes of acute Achilles tendon rupture with operative or nonoperative treatment using an identical functional bracing protocol. Foot Ankle Int. 32(4): p. 337-43. PMC 21733434
4. Nilsson-Helander, K., et al., Acute achilles tendon rupture: a randomized, controlled study comparing surgical and nonsurgical treatments using validated outcome measures. Am J Sports Med. 38(11): p. 2186-93. PMC 20802094
5. Willits, K., et al., Operative versus nonoperative treatment of acute Achilles tendon ruptures: a multicenter randomized trial using accelerated functional rehabilitation. J Bone Joint Surg Am. 92(17): p. 2767-75. PMC 21037028
6. Kongsgaard, M., et al., Structural Achilles tendon properties in athletes subjected to different exercise modes and in Achilles tendon rupture patients. J Appl Physiol, 2005. 99(5): p. 1965-71. PMC 16081623
7. Molloy, A. and E. V. Wood, Complications of the treatment of Achilles tendon ruptures. Foot Ankle Clin, 2009. 14(4): p. 745-59. PMC 19857846
8. Thompson, J. and B. Baravarian, Acute and chronic Achilles tendon ruptures in athletes. Clin Podiatr Med Surg. 28(1): p. 117-35. PMC 21276522
9. Parekh, S. G., et al., Epidemiology and outcomes of Achilles tendon ruptures in the National Football League. Foot Ankle Spec, 2009. 2(6): p. 283-6. PMC 20400426
10. Arocho, A et al Validation of the 2-[DELTA][DELTA]Ct Calculation as an Alternate Method of Data Analysis for Quantitative PCR of BCR-ABL P210 Transcripts. Diagnostic Molecular Pathology March 2006—Volume 15—Issue 1—pp 56-61

### Example 5

To further clarify the decellularization and particle size reduction process, the following experiment was performed.

A human placenta was processed similar to the decellularization and particle size reduction process of Example 2 through the grinding, pH adjustment and H202 addition steps except for the following changes.

The weight of the placenta on removal from the freezer was 605 grams.
The cut up placenta was homogenized with a Waring type blender.
The solids content of the ground unprocessed tissue material was 5%.
After the H202 addition the slurry was mixed for an hour prior to filtration.

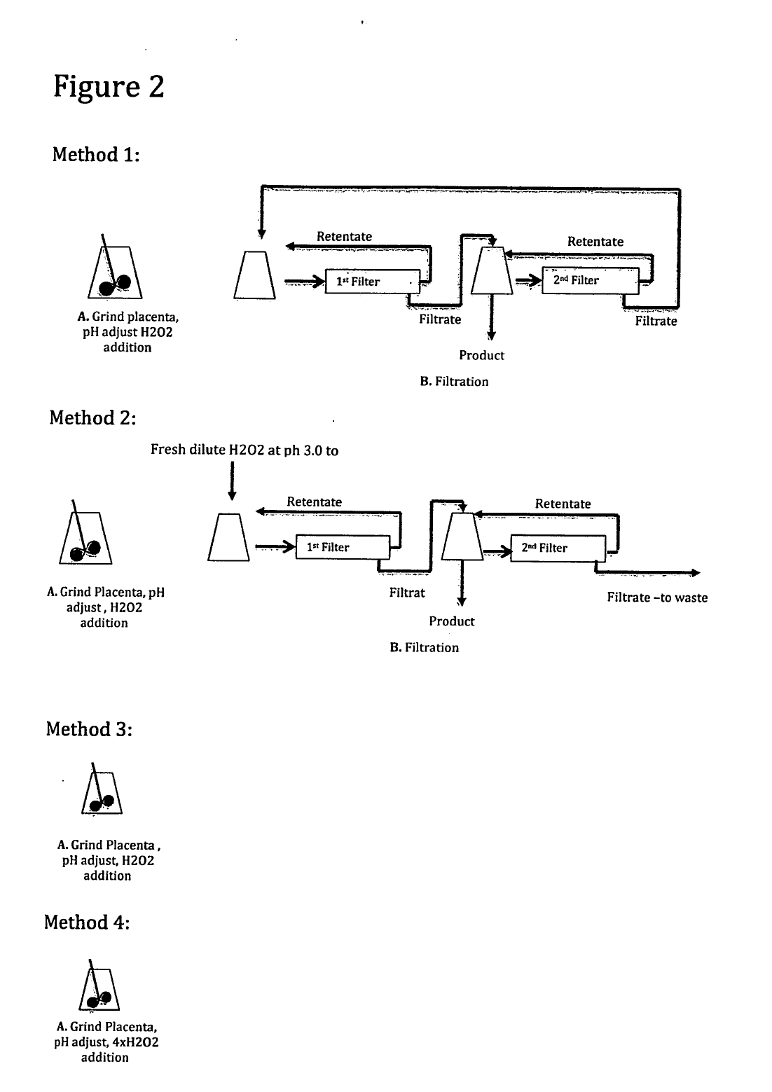

After the H2O2 addition the slurry was split into 4 separate aliquots to examine 4 different methods of decellularization (FIG. 2) and particle size reduction.

Method 1: (Recycle) The filtration process was followed to completion.

Method 2: (No recycle) The process was similar to example 1 except instead of passing the 2^{nd} filter filtrate back to the pre-first filter slurry it was discarded. To replace the discarded 2 filter filtrate, fresh sterile H2O that was adjusted to pH 3.0 to 3.3 and that contained the same amount H2O2 per liter that was used in method 1 was added at the same rate the 2^{nd} filter filtrate was discarded.

Method 3: (Batch) The process was completed as a batch process with no filtration. The slurry was mechanically agitated for 3 hours and then lyophilized.

Method 4: (Batch 4) This was also completed as a batch process except 4× the H2O2 was added to the slurry then it was mechanically agitated for 3 hours and then lyophilized. The process was stopped for all 4 methods after the first lyophilization so no finish processing was completed. The dry product was then washed with a 100 KD centrifuge filter to simulate finish processing before being analyzed for protein and DNA content.

The following results were obtained.

Tissue Yield: Method 3 (batch) and Method 4 (batch) gave tissue yields close to that of the 100% raw control, Method 1 (recycle) gave a tissue yield of about 80%, while Method 2 (no recycle) gave a yield of only about 40% of that of the raw placenta.

Collagen yield: The “batch” method and “batch 4” method gave yields of about 97% and 77% respectively while the “recycle” and “no recycle” gave yields of about 43% and 26% respectively, all compared to the raw placenta collagen content.

The “recycle” method and the “batch” method resulted in the highest yields of fibronectin with respectively 77% and 81% of that of the raw placenta control while the “no recycle” and “batch 4” methods retained 37% and 35% of fibronectin respectively. The “recycle” method had the highest yield of Laminin of 5.5% followed by the “no recycle” method at 2.6%. The “batch” methods retain virtually no Laminin.

All the methods reduced DNA content significantly from the raw placenta with the “recycle” and “batch 4” methods reducing the concentration of DNA the most with 79% and 81% respectively. The “no recycle” method was the next most efficient reducing the DNA concentration by 65% and the “batch” method was the least efficient reducing the DNA concentration by 54%.
The results of this example show that the “recycle method” is the most efficient in reducing DNA concentration while maintaining the maximum amount of protein yield. It also shows the “batch” methods as being the most harmful to the more delicate proteins like Laminin.

### Example 6

A human placenta was processed similar to the decellularization and particle size reduction process of Example 2 and FIG. 1 except for the following changes.

The weight of the placenta on removal from the freezer was 504 grams.

The cut up placenta was homogenized with a Waring type blender.
The ground tissue was re-suspended to 4 liters before the solids content was sampled for measurement and the solids content was 1.07%.
The first filter had a pore size of 55 micron not 100 micron.
The process was stopped after the first lyophilization so no finish processing was completed. The dry product was then washed with a 100 KD centrifuge filter to simulate finish processing before being analyzed for protein and DNA content.

The resulting process and product had the following characteristics:

After filtration, approximately 90% of the starting material was captured as 2^{nd} filter retentate.
The DNA concentration of the product showed a reduction of approximately 80% from pre-processed thawed and ground placenta material.
The collagen content of the product as a percentage of dry material was equal to approximately 63% which is equal to 76% of the collagen content of pre-processed thawed and ground placenta as a percentage of dry material.
The DNA content of the product as a percentage of dry material was approximately 0.28% which is equal to an approximately 69% reduction of the DNA content of pre-processed thawed and ground placenta as a percentage of dry material.
This Example shows that a using a 55 micron, 1^{st} filter in the filtration process of Example 2 and 3 results in approximately the same tissue yield, protein yield and DNA concentration reduction to that of using 100 micron first filter as compared to the results of Example 3.

### Example 7

A human placenta was processed similar to the decellularization and particle size reduction process of Example 2 and FIG. 1 except for the following changes.

The weight of the placenta on removal from the freezer was 587 grams.

The cut up placenta was homogenized with a Waring type blender.
The ground tissue was re-suspended to 2 liters before the solids content was sampled for measurement and the solids content was 3.4%.
No H2O2 was added to the ground placenta slurry and it was only subject to a pH adjustment to approximately 3.2 as a means of decellularization and particle size reduction
The first filter had a pore size of 55 micron not 100 micron.
The material was washed and neutralized but none of the other finish processing steps were completed.

The resulting process and product had the following characteristics:

After filtration approximately 32% of the starting material was captured as 2^{nd} filter retentate The DNA concentration of the product showed very little reduction from pre-processed thawed and ground placenta material at 20%.

This Example shows that using the filtration process of Example 2 without using a reagent for decellularization and particle size reduction results in a very low yield of product with no effective decellularization.

### Example 8

A human placenta was processed similar to the decellularization and particle size reduction process of Example 2 and FIG. 1 except for the following changes.

The weight of the placenta on removal from the freezer was 542 grams.

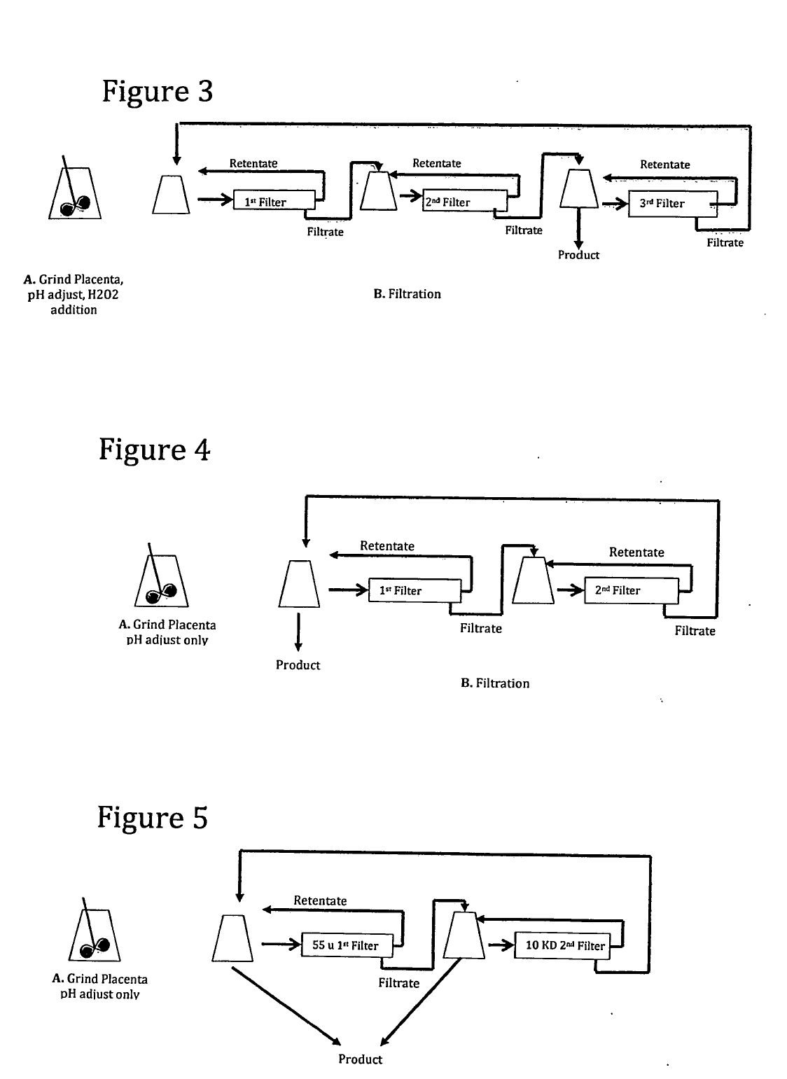

The cut up placenta was homogenized with a Waring type blender.
The ground tissue was re-suspended to 4 liters before the solids content was sampled for measurement and the solids content was 1.53%.
The first filter had a pore size of 55 micron not 100 micron.
The second filter had a pore size of 0.5 micron not 100 micron.
A third filter with a pore size of 10 KD was added at the end of the process with the product coming from the 10 KD retentate (FIG. 3).
The process was stopped after the first lyophilization so no finish processing was completed.
The resulting process and product had the following characteristics:
After filtration approximately 64% of the starting material was captured as 2^{nd} filter retentate. The collagen content of the product as a percentage of dry material was equal to approximately 58% which is equal to 93% of the collagen content of pre-processed thawed and ground placenta as a percentage of dry material. The DNA content of the product as a percentage of dry material was approximately 0.02% which is equal to an approximately 98% reduction of the DNA content of pre-processed thawed and ground placenta as a percentage of dry material.

This example shows that this process can reduce the particle size of the placental tissue so as to pass through a 0.5 micron filter while although sacrificing some overall tissue yield still retaining a high level of collagen, fibronectin and laminin concentration and very effectively reducing the DNA content of the raw placental tissue.

### Example 9

A human placenta was processed similar to the decellularization and particle size reduction process of Example 2 and FIG. 1 except for the following changes.

The weight of the placenta on removal from the freezer was 426 grams.

The cut up placenta was homogenized with a Waring type blender.
The ground tissue was re-suspended to 2 liters before the solids content was sampled for measurement and the solids content was 2.35%.
The first filter had a pore size of 55 micron not 100 micron.
The product was taken from the 55 micron retentate not the 10 KD retentate (FIG. 4).
The product washed using the first filter by re-suspending the 55 micron retentate to 1 liter with sterile H2O, filtering it down to approximately 250 mls and repeating three times. The 55 micron retentate was then neutralized and lyophilized. No other finish processing steps were completed.

The resulting process and product had the following characteristics:

The collagen content of the product as a percentage of dry material was equal to approximately 50% which is equal to 80% of the collagen content of pre-processed thawed and ground placenta as a percentage of dry material.
The DNA content of the product as a percentage of dry material was approximately 0.05% which is equal to a 85% reduction of the DNA content of pre-processed thawed and ground placenta as a percentage of dry material.
The lyophilized product was much stronger than previous examples where the product was taken from the 10 KD retentate/55 micron filtrate slurry only.

This example shows a product that results from the decellularization and particle size reduction process that has the characteristics of being very strong and that may have uses as a bulking agent or as an occlusive wound dressing that provides a barrier to prevent infection to the wound.

### Example 10

A human placenta was processed similar to the decellularization and particle size reduction process of Example 9, FIG. 4 except for the following changes.

The product was taken from mixing the 55 micron retentate and the 10 KD retentate (FIG. 5) prior to the first lyophilization and after the 10 KD retentate had been neutralized and washed. The mixed product was then lyophilized and pressed into a pad.

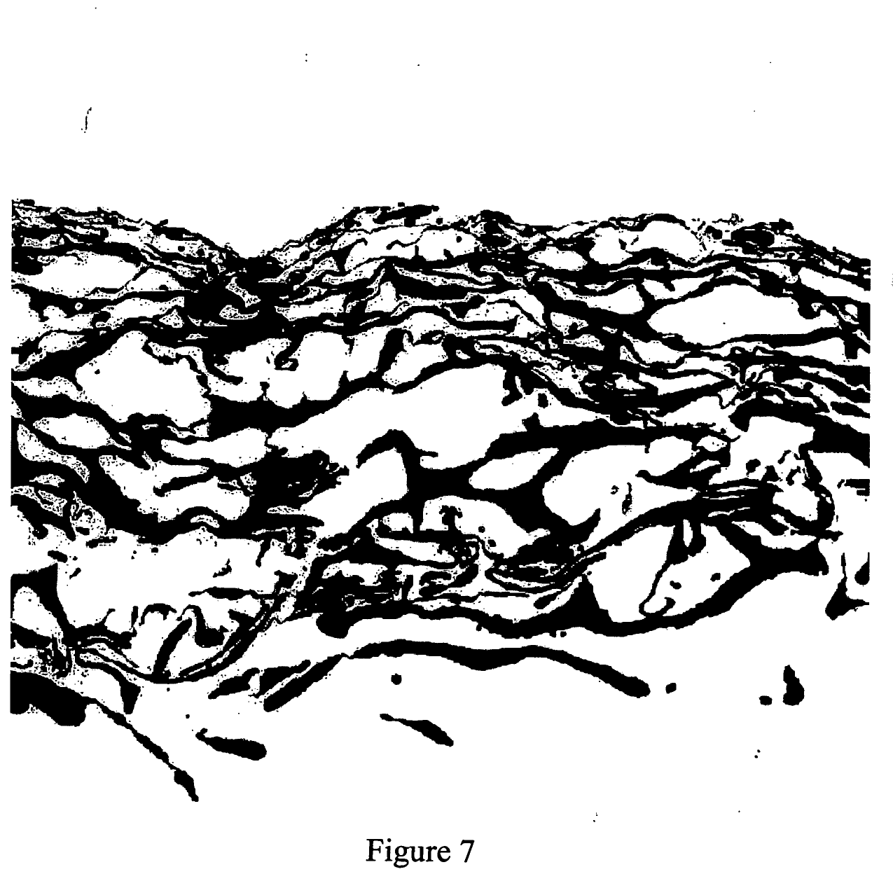

The resulting process and product had the following characteristics:
The lyophilized product had heterogeneous architecture (FIG. 7) with the larger 55 micron retentate particles mixed in with the finer 10 KD retentate particles.
The lyophilized product was much stronger than previous examples where the product was taken from the 10 KD retentate/55 micron filtrate slurry only.

This example shows a product that results from the decellularization and particle size reduction process that not only has the characteristics of being stronger than product taken from the 10 KD retentate/55 micron filtrate slurry only but still maintains the open channels of the 10 KD retentate/55 micron slurry that allow for cellular penetration in a wound bed or any other instance where cell penetration into the product is desired.

### Example 11

A human placenta was processed similar to the decellularization and particle size reduction process of Example 10 FIG. 5 except for the following changes.

Rather than mixing the 55 micron retentate and the 10 KD retentate prior to the second lyophilization, the 55 micron material was poured as a sheet and then frozen to −70 degrees C. Then the 10 KD retentate material that was chilled to the temperature range of 2-4 degrees centigrade was poured over the top of the frozen 55 micron retentate material and the product was lyophilized pressed into a sheet and cut into 3×3 cm pieces. No other finish processing steps were completed.

The resulting process and product had the following characteristics:

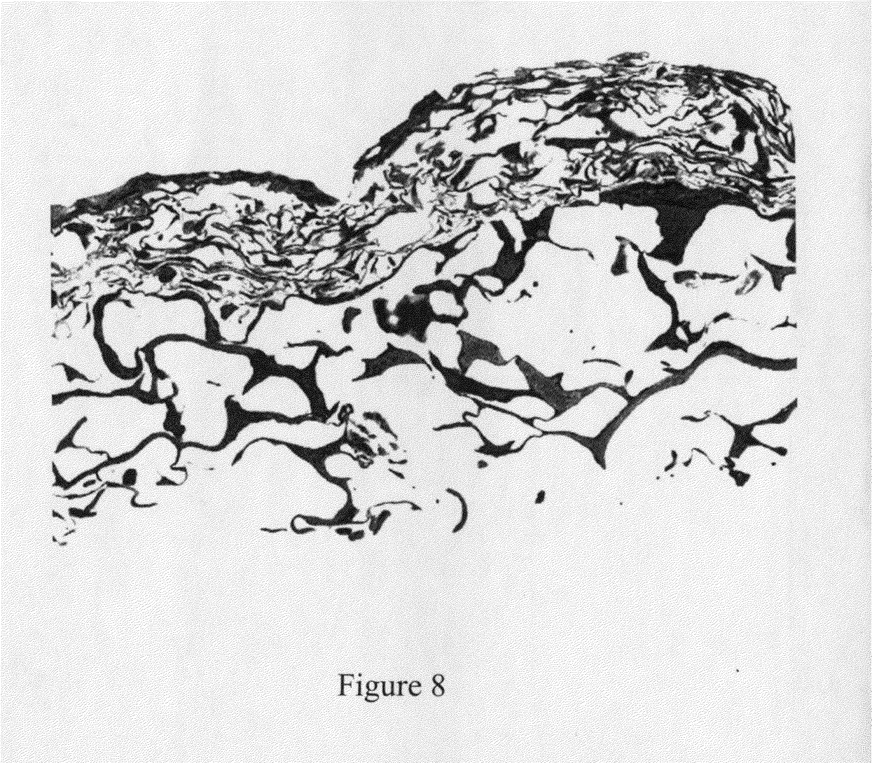

The lyophilized product had a bi-layered architecture (FIG. 8) which when flipped over after preparation has the larger 55 micron retentate particles layered over the smaller 10 KD retentate particles. It is the smaller particle layer which will come into contact with the wound bed when the by-layered product is used for wound healing purposes.
The lyophilized product is stronger than previous examples where the product was taken from the 10 KD retentate/55 micron filtrate slurry only.

This example shows a product that results from the decellularization and particle size reduction process that not only has the characteristics of being stronger than product taken from the 10 KD retentate/55 micron filtrate slurry only but still maintains the open channels of the 10 KD retentate/55 micron slurry that allow for cellular penetration in a wound bed or any other instance where cell penetration into the product is desired and provides an occlusive layer that provides a barrier to prevent infection in a wound bed or any other instance where an occlusive outer layer would be desired.

While the by-layered product presented in this Example 11 presents the upper layer with the 55 micron particle size and the lower layer (i.e., the layer in contact with the wound bed), as the smaller 10 KD retentate particle size, it should be appreciated that in this aspect of the invention, the by-layered product is not limited to the sizes set forth in the Example. In particular, this aspect of the invention can be characterized as a by-layered construct which comprises two layers of disparate particle sizes wherein one layer, the lower layer relative to the horizontal plane, is intended to, in one aspect, come into contact with a wound bed, and has the smaller average particle size than a top layer which is comprised of the larger particle size. The top layer is preferably of a particle size which passes through 10 to 200 micron filter pores and the lower layer preferably of a particle size which passes through sub-micron pores preferably below 0.2 microns down to 10 KD or below. Stated in more general terms, the product is a by-layered construct comprising relative to the horizontal plane, a top layer comprising an upper surface and a lower surface, and a lower layer comprising a top surface and a lower surface wherein the lower surface of the top layer is in contact with the top surface of the lower layer, and each layer is a decellularized small particle tissue derived from biological material that has previously been treated with a decellularizing agent and submitted to particle size separation stages such that the particle sizes of the top layer are larger than the particle sizes of the lower layer.

### Example 12

A human placenta was processed similar to the decellularization and particle size reduction process of Example 2 and FIG. 1 except for the following changes.

Chicken bone (instead of human placenta) was subject to the decellularization and particle size production process. The skin, meat and cartilage from 12 chicken drumsticks were removed from the bone. The chicken bone was chopped into pieces with the resulting weight being 561 grams of bone and marrow. The chicken bone pieces were then resuspended in 1800 mls of sterile water.

The mixture was adjusted to a pH of approximately 3.05 with 2N HCL and 2N NaOH if required and allowed to sit at 2 to 10 degrees C. for approximately 72 hours.

The chopped bone slurry was then ground in a meat grinder. Additional sterile water was used to wash the bone through as it was being ground. The resulting ground slurry was approximately 4 liters and had a solids content of approximately 4.5%. A sample was taken of this material as a representation of pre-processed bone for further protein content and DNA content analysis.

The slurry was pH adjusted to approximately 3.05 with 2N HCL and 2N NaOH if required.

The first filter had a pore size of 55 micron not 100 micron and the second filter had a pore size of 50 KD not 10 KD.
The process was stopped after the first lyophilization so no finish processing was completed. The dry product and pre-processed sample were then washed with a 100 KD centrifuge filter to simulate finish processing before being analyzed for protein and DNA content.

The resulting process and product had the following characteristics:

After filtration approximately 45% of the starting material was captured as 2^{nd} filter retentate and 33% of the starting material was captured as 1^{st} filter retentate.
The DNA content of the product as a percentage of dry material was approximately 0.05% which is equal to a 24% reduction of the DNA content of pre-processed bone as a percentage of dry material.
The BMP-2 content of the product as a percentage of dry material was equal to approximately 51% of the BMP-2 content of pre-processed bone as a percentage of dry material.

This example shows that the decellularization and particle size reduction process has utility in processing bone into small, decellularized particles which can be used in any procedure where decellularized bone is used.

What is claimed is:

1. A process for producing a decellularized small particle tissue which comprises the following steps:
(a) Selecting an appropriate tissue starting material from which a decellularized small particle tissue is desired to be prepared,
(b) treating the tissue with a decellularizing agent at an acid pH to remove at least a portion of the cellular material therefrom and to yield a product comprising a liquid component and a solid component,
(c) subjecting the liquid component and the solid component to a plurality of filters, F₁ through Fₙ, wherein n may be 2, or an integer higher than 2, wherein the pore sizes of the filters F₁ through Fₙ range from 10 Kilo Daltons to 200 microns, yielding filtrates and retentates,
(d) recycling either of said filtrates or said retentates or both, either separately or together, to any of steps b) or c) or both, at least one time,
(e) isolating a decellularized small particle tissue from any of steps b), c) or d).

2. The process of claim 1 wherein a) is a blood-laden placenta or a blood-laden tissue.

3. The process of claim 1 wherein b) is an oxidizing agent.

4. The process of claim 2 wherein b) is hydrogen peroxide.

5. The process of claim 4 wherein the plurality in step c) is at least 2 filters.

6. The process of claim 5 wherein the pore size of any filter prior to the sequentially last stage filter is between 10 Kilo Daltons to 200 microns.

7. The processes of claim 5 wherein the pore size of the sequentially last stage filter, is between 10 Kilo Dalton-0.2 microns.

8. The process of claim 1 wherein the pH of step b) is from 2-7.

9. The process of claim 2 wherein the tissue in step a) is homogenized.

10. The process of claim 9 wherein the reaction product in step b) is homogenized,

11. The process of claim 1 wherein filtrate from any filter that is not the sequentially last stage filter (smallest pore size) is passed to the next filter that has the sequentially smaller pore size.

12. The process of claim 1 wherein retentate from any filter is passed back to the decellularization and size reduction vessel or combined with the filtrate of any upstream filter or both.

13. The process of claim 1 wherein filtrate from the sequentially last stage (smallest pore size) is recycled to any filter or to the decellularizing and size reduction step a) or both.

14. The process of claim 1 wherein the pH in step b) may be adjusted to be less acidic.

15. The process of claim 1, wherein said tissue decellularizing or particle size separation or both are conducted at from 10 to 20 degrees centigrade.

16. The process of claim 1, wherein said decellularizing agent is an oxidizing agent employed after the tissue mixture has been pH adjusted to between 2 and 7.

17. The process of claim 1, wherein said particle size separation system is comprised of a series of three filters 100 micron, 0.2 micron and 10 Kilo Daltons respectively and the product is taken from the stage ranging from 10 Kilo Daltons to 0.2 micron.

18. The process of claim 1, wherein said decellularizing reaction or particle size separation system or both is aided by sonication frequencies between 10 and 50 kHz.

19. The process according to claim 1, wherein prior to step e), the pH is adjusted to between 6 and 8.

20. The process according to claim 1, wherein step e) includes lyophilizing the small particle tissue product.

21. A process for producing a decellularized small particle tissue which comprises the following steps:
(a) Selecting an appropriate tissue starting material from which a decellularized small particle tissue is desired to be prepared,
(b) treating the tissue with a decellularizing agent at an acid pH to remove at least a portion of the cellular material therefrom and to yield a product comprising a liquid component and a solid component,
(c) subjecting the liquid component and the solid component to a plurality of particle separation stages, wherein the separation stages are capable of separating successively smaller particle sizes from the previous stage, yielding a liquid component and a solid component,
(d) recycling either of said liquid or solid components or both, either separately or together, to any of steps b) or c) or both, at least one time,
(e) isolating a decellularized small particle tissue from any of steps b), c) or d).

22. A decellularized small particle tissue derived from biological material that has previously been treated with a decellularizing agent, which comprises the following constituents,
as a percentage by weight of the dry starting biomaterial tissue concentration:
Collagen, at least 40% and preferably from 60% to 70%;
Elastin, at least 50% and preferably from 70% to 90;
Laminin, at least 10% and preferably from 12% to 21%;
Fibronectin, at least 30% and preferably from 40% to 60%;
ds DNA of less than 10% to 15% and preferably less than 5%; and

23. A decellularized small particle tissue derived from biological material that has previously been treated with a decellularizing agent, which comprises the following constituents,
as a percentage of the dry weight of the final biomaterial product concentration:
Collagen of at least 30% and preferably from 40% to 50%,
Elastin of at least 2% and preferably from 4% to 5%,
Laminin of at least 0.025% and preferably from 0.045% to 0.01%,
Fibronectin of at least 0.10% and preferably from 0.12% to 0.3%,
Glycosaminoglycan of at least 0.2% and preferably from 0.3% to 0.5%,
ds DNA less than 0.1%, and
Endotoxin less than 0.1% EU/mg.
